(19) United States
(12) Patent Application Publication
(10) Pub. No.: US 2009/0012323 A1
(43) Pub. Date: Jan. 8, 2009

(54) PRODUCTION OF DETERGENT RANGE ALCOHOLS

(75) Inventors: Hendrik Van Rensburg, Fife (GB); Craig McGregor, District Parys (ZA)

Correspondence Address: INDIANAPOLIS OFFICE 27879;BRINKS HOFER GILSON & LIONE, ONE INDIANA SQUARE, SUITE 1600, INDIANAPOLIS, IN, 46204-2033, US

(73) Assignee: Sasol Technology (Proprietary) Limited, Rosebank (ZA)

(21) Appl. No.: 12/164,852

(22) Filed: Jun. 30, 2008

(30) Foreign Application Priority Data
Jul. 2, 2007 (ZA) 2007/04830

(51) Int. Cl. C07C 31/125; C07C 203/00

(52) U.S. Cl. 558/488; 568/909

(57) ABSTRACT

This invention relates to a process for the production of a mixture of detergent-range alcohols having an average of between 8 and 20 carbon atoms per molecule. The process includes the steps of providing a hydrocarbon stream containing olefins and paraffins in which more than 5% by volume of olefin molecules in the hydrocarbon stream have a total number of carbon atoms which is different from the total number of carbon atoms of the most abundant two carbon numbers of olefins in the hydrocarbon stream; reacting the hydrocarbon stream with CO and an alcohol in the presence of a catalyst in a hydroesterification reaction to form a hydrocarbon stream containing esters and paraffins; separating esters from the hydrocarbon stream containing esters and paraffins; and subjecting the esters to a hydrogenation reaction to provide the mixed alcohol product.

### BACKGROUND OF THE INVENTION

THIS invention relates to a process for the production of detergent range alcohols.

Detergent range alcohols are alcohols, usually in the C₈ to C₂₀ range (i.e. from 8 to 20 carbon atoms in the molecules), that are useful in the manufacture of detergents and surfactants.

One method of producing such detergent range alcohols is to dimerise a short chain olefin feedstock, e.g. 1-Hexene to a longer chain olefin e.g. 1-dodecene, followed by hydroformylation to tridecanol. In this case separation of paraffins with close boiling points (e.g. hexane) in the 1-Hexene feedstock is relatively simple since the boiling point of hexane and tridecanol differs considerably.

Detergent alcohols are also produced commercially by distilling a desired cut of olefins and paraffins from an olefin-rich feed having a wide range of carbon atoms, e.g. those produced by the Fischer Tropsch process. The olefins are then converted to alcohols by hydroformylation. Paraffins are inert to the hydroformylation reaction and form part of the product. Because it is difficult to separate the alcohols from paraffins in the product where the boiling points of the alcohols and paraffins are similar or overlap, it is an accepted practice to distill a narrow-cut of olefins containing a high concentration of two or less carbon numbered olefins (2C range olefins) and its associated paraffins. In the case of 2C range olefin feedstocks, conversion of the olefins to alcohols increases the boiling points of the olefins relative to the paraffins, thereby facilitating easy separation of the alcohols from the associated paraffins.

However, from an economical point of view it is not always possible to use narrow-cuts of olefins especially where the volumes of feedstock available is too small to attain the economy of scale required for the alcohol production process. In these cases it may be desirable to increase the volumes of the olefin feedstock by separating a broader cut of olefins having more than two carbon number olefins (3C or greater). However the separation problem discussed above complicates this option.

It is an object of this invention to provide an improved method for the production of alcohols, typically used as detergent precursors.

### SUMMARY OF THE INVENTION

According to an aspect of the invention there is provided a process for the production of a mixture of alcohols, typically a mixture of detergent-range alcohols having an average of between 8 and 20 carbon atoms per molecule, including the steps of:
1) providing a hydrocarbon stream containing olefins and paraffins in which more than 5% by volume of olefin molecules in the hydrocarbon stream have a total number of carbon atoms which is different from the total number of carbon atoms of the most abundant two carbon numbers of olefins in the hydrocarbon stream;
2) reacting the hydrocarbon stream with CO and an alcohol in the presence of a catalyst in a hydroesterification reaction to form a hydrocarbon stream containing esters and paraffins;
3) separating esters from the hydrocarbon stream containing esters and paraffins; and
4) subjecting the esters to a hydrogenation reaction to provide an alcohol product.

Preferably, the hydrocarbon stream in step 1) contains olefins in which more than 10%, more preferably more than 20%, typically more than 40%, by volume, of olefin molecules in the feed have a total number of carbon atoms which is different from the total number of carbon atoms of the most abundant two olefins (by carbon number) in the hydrocarbon stream.

The hydrocarbon stream in step 1) may contain olefins in which more than 5%, possibly more than 10% by volume, of olefin molecules in the feed have a total number of carbon atoms which is different from the total number of carbon atoms of the most abundant three, four or five olefins (by carbon number) in the hydrocarbon stream.

The hydrocarbon feed stream in step 1) may have an average number of carbon atoms per molecule of from 10 to 18.

The hydrocarbon feed stream in step 1) may contain olefins in predominantly a 3C range or greater, typically predominantly in a 4C range, such as C₁₁-C₁₄ olefins, or a 6C range, such as C₁₀-C₁₅ olefins. A 3C range means that the olefins are present in a Cₓ-Cₓ₊₂ range, a 4C range means that the olefins are in a Cₓ-Cₓ₊₃ range, and a 6C range means that the olefins are in a Cₓ-Cₓ₊₅ range; where x may be any number from 5 to 20, typically from 10 to 15. Preferably, more than 80%, more preferably more than 90%, by volume, of the olefins are in the range.

Preferably, the hydrocarbon feed stream in step 1) is derived from a Fischer-Tropsch condensate product.

Oxygenates may be removed from the hydrocarbon feed stream, prior to subjecting the stream to the hydroesterification reaction of step 2).

The hydrocarbon stream containing esters and paraffins (in steps 2 and 3) may also contain olefins.

Esters may be separated from the hydrocarbon stream containing esters, olefins and paraffins in step 3) by distillation.

The hydroesterification reaction of step 2) is typically carried out in the presence of a catalyst comprising cobalt and a nitrogen-containing additive such as pyridine.

The hydroesterification reaction of step 2) is generally performed at temperatures between 120-170° C. and CO pressures between 80-150 bar (8-15×10⁶ Pa).

### BRIEF DESCRIPTION OF THE DRAWINGS

FIG. 1 is a flow diagram of a process of the invention;

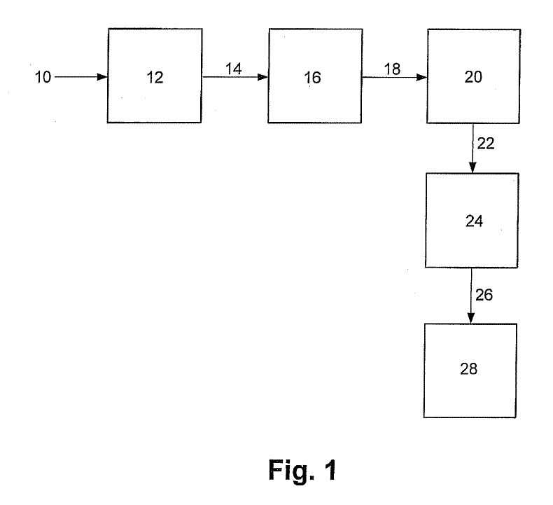

FIG. 2 is a GC trace of a reaction mixture of C₁₀-C₁₅ paraffins and C₁₁-C₁₆ butyl esters from Example 1a;

FIG. 3 is a GC trace of the reaction mixture of FIG. 2 after fractional distillation removing paraffins from butyl esters;

FIG. 4 is a GC trace of the mixture of FIG. 3 after reduction of the esters to the corresponding C₁₁-C₁₆-alcohols;

FIG. 5 is a GC trace of a reaction mixture of hydroformylation of C₁₀-C₁₅ paraffins/α-olefins to the corresponding C₁₁-C₁₆ alcohols from Comparative Example 1b;

FIG. 6 is a GC trace of Fischer-Tropsch derived C₁₁-C₁₄ olefins and paraffins from Example 2;

FIG. 7 is a GC trace of a reaction mixture of Co/Py/BuOH hydroesterification of C₁₁-C₁₄ Fischer-Tropsch derived olefins/paraffins to C₁₂-C₁₅ butyl esters from Example 2a;

FIG. 8: is a GC trace of pure C₁₂-C₁₅ butyl esters after fractional distillation removal of residual C₁₁-C₁₄ paraffins from the mixture of FIG. 7;

FIG. 9 is a GC trace C₁₂-C₁₅ alcohols produce from LiAlH₄ reduction of the corresponding C₁₂-C₁₅ butyl esters of FIG. 8; and

FIG. 10 is a GC trace of a reaction mixture of hydroformylation of Fischer-Tropsch derived C₁₁-C₁₄ olefins/paraffins paraffins to the corresponding C₁₂-C₁₅ alcohols from Comparative Example 2b.

### DESCRIPTION OF PREFERRED EMBODIMENTS

This invention relates to the production of detergent range alcohols (usually in the C₈ to C₂₀ range, i.e. from 8 to 20 carbon atoms in the molecules) from a hydrocarbon feedstream. The feedstream is preferably from a Fischer-Tropsch process.

A typical Fischer-Tropsch process involves the hydrogenation of CO in the presence of Group VIII metals, and includes, but is not limited to, Fe, Co, Mo, W, Rh, Pt, Pd Os, Ir and Ru. In principle, iron-based, cobalt-based or iron/cobalt-based Fischer-Tropsch catalysts can be used commercially in the Fischer-Tropsch reaction stage. Iron based catalysts are preferred for the present invention since they tend to produce a more olefinic hydrocarbon condensate product. In some embodiments, the iron-based Fischer-Tropsch catalyst may comprise iron and/or iron oxides which have been precipitated or fused. However, iron and/or iron oxides which have been sintered, cemented, or impregnated onto a suitable support may also be used. In some cases the iron based catalyst may contain various levels of promoters, the role of which may be to alter one or more of the activity, the stability, and the selectivity of the final catalyst. Preferred promoters are those influencing the surface area of the reduced iron (‘structural promoters’), and these include oxides or metals of Mn, Ti, Mg, Cr, Ca, Si, Al, or Cu or combinations thereof. Preferred promoters for influencing product selectivities are alkali oxides of K and Na. Catalysts for the production of hydrocarbon species by a Fischer Tropsch process are generally known in the art.

The Fischer-Tropsch reaction may be effected in a fixed bed or preferably in a slurry phase reactor for low temperature Fischer-Tropsch applications, or in a fluidized bed reactor for high temperature Fischer-Tropsch applications. The Fischer-Tropsch reaction conditions may include utilizing a reaction temperature of between 190° C. and 340° C., with the actual reaction temperature being largely determined by the desired product spectrum. The products formed from this reaction are gaseous, liquid and optionally waxy hydrocarbons that include, inter alia, olefins and paraffins as well as oxygenates. The carbon number distribution of these products is generally described by the Anderson-Schulz-Flory distribution.

The low temperature Fischer-Tropsch (LTFT) process is a well known process in which synthesis gas, a mixture of gases including carbon monoxide and hydrogen, is reacted over a suitable catalyst to produce a mixture of straight and branched chain hydrocarbons ranging from methane to waxes with molecular masses above 1400 and smaller amounts of oxygenates (relative to high temperature Fischer-Tropsch). The LTFT catalyst may comprise active metals such as iron, cobalt, nickel or ruthenium and the catalyst will normally be a precipitated or supported catalyst. Synthesis gas for the LTFT process may be derived from any carbon containing feedstock such as coal, natural gas, biomass or heavy oil streams. Some reactors for the production of heavier hydrocarbons using the LTFT process are slurry phase or tubular fixed bed reactors, while operating conditions are generally in the range of 180-280° C., in some cases in the 210-260° C. range, and 10-50 bar, in some cases between 20-30 bar. The molar ratio of hydrogen to carbon monoxide in the synthesis gas may be between 0.4 and 3.0, generally between 1.0 and 2.0.

As is the case with the LTFT process, the High Temperature Fischer-Tropsch (HTFT) process also makes use of the FT reaction albeit at a higher process temperature. A typical catalyst for HTFT process is iron based. Fused iron catalysts are best known in the prior art to be used in high temperature Fischer-Tropsch synthesis. Known reactors for the production of heavier hydrocarbons using the HTFT process are the circulating bed system or the fixed fluidized bed system, often referred to in the literature as Synthol processes. These systems operate at temperatures in the range 290-360° C., and typically between 320-350° C., and at pressures between 20-50 bar, in some cases between 20-30 bar. The molar ratio of hydrogen to carbon monoxide in the synthesis gas is essentially between 1.0 and 3.0, generally between 1.5 and 2.5. Generally, recycle streams are applied to increase the hydrogen content in the feed gas to the HTFT reactor to give a molar ratio of hydrogen to carbon dioxide at the reactor inlet of between 3.0 and 6.0 depending on the amount of carbon dioxide in the feed gas. Generally, a stoichiometric ratio, known as the Ribblett ratio H₂/[2(CO)+3(CO₂)]=1.03 is used as a target for the feed gas composition. Products from the HTFT process are all in the vapour phase at the reactor exit and are somewhat lighter than those derived from the LTFT process and, as an additional distinction, contain a higher proportion of unsaturated species and oxygenates. The HTFT process is completed through various steps which include the preparation of synthesis gas (H₂ and CO) from any carbon containing feedstock such as by natural gas reforming or gasification of coal or other suitable hydrocarbonaceous feedstocks like petroleum based heavy fuel oils or biomass. This is followed by the HTFT conversion of synthesis gas in a reactor system like the Sasol Synthol or the Sasol Advanced Synthol reactor. One of the products from this synthesis is an olefinic distillate, also known as Synthol Light Oil (SLO).

Detailed descriptions of these two FT processes, LTFT and HTFT, may be found in “Fischer-Tropsch Technology”, Studies in Surface Science and Catalysis, Vol. 152, Eds. A. P. Steynberg and M. E. Dry, Elsevier, 2004, amongst others.

With reference to the Drawing, in accordance with the invention, a hydrocarbon condensation product of a LTFT or HTFT Reaction 10 is fractionated 12 to provide a hydrocarbon stream 14 containing olefins in which more than 5%, preferably more than 10%, more preferably more than 20%, typically more than 40%, by volume, of olefin molecules in the hydrocarbon stream have a total number of carbon atoms which is different from the total number of carbon atoms of the most abundant two carbon numbers of olefins in the hydrocarbon stream. The fractionated hydrocarbon stream 14 may contain carbons in predominantly a 3C range or greater, typically in predominantly a 4C range, such as a C₁₁-C₁₄, or in a 6C range such as C₁₀-C₁₅. The fractionated hydrocarbon stream is introduced to an oxygenate removal step 16 in which oxygenates are removed from the stream. The oxygenate removal step may be achieved by using Liquid-Liquid Extraction (e.g. Acetonitrile/water or methanol/water), dehydration or hydrogenation.

From the oxygenate removal step 16, a fractionated hydrocarbon feed stream 18 containing paraffins and olefins is subjected to a hydroesterification reaction 20 to produce a stream 22 containing esters, paraffins and unreacted olefins. In the hydroesterification reaction 20, olefins in the stream 18 are reacted with CO and an alcohol such as methanol or ethanol, and a catalyst comprising cobalt and a nitrogen-containing additive such as pyridine to produce a wide range of alkoxy esters. In the case of C₁₁-C₁₄ olefins, C₁₂-C₁₅ alkyl esters are produced and in the case of C₁₀-C₁₅ olefins, C₁₁-C₁₆ alkyl esters may be produced.

In a preferred embodiment of the invention, the hydroesterification reaction 20 is carried out in the presence of a catalyst comprising cobalt and pyridine. The cobalt/pyridine catalyzed hydroesterfication reaction allows for the production of a wide range of alkoxy esters and thus a primary alcohol may be selected such that the resulting esters have boiling points that do not overlap with residual paraffins and alkenes in order to allow for separation of the esters by distillation. The hydroesterification reaction 20 is generally performed at temperatures between 120-200° C. and CO pressures between 80-150 bar. Pyridine acts as a catalytic promoter in this reaction, enhancing both activity and selectivity to the desired linear products. The preferred Pyridine/Cobalt ratio is 20:1 or greater, typically from 20:1 to 40:1, preferably from 24:1 to 40:1, most preferably 32:1.

The resulting esters have boiling points which do not overlap with the residual olefins and paraffins and can be separated from the olefins and paraffins in a distillation step 24.

Esters 26 separated during the distillation step 24 may be converted to detergent range alcohols in a hydrogenation reaction 28.

The hydrogenation reaction 28 may be carried out using a copper-containing catalyst at temperatures in the range of 200 to 300° C. and pressures between 20 and 30 MPa. Liquid-phase hydrogenation of free fatty acids is used industrially, although catalysts and equipment have to meet higher demands due to corrosion and catalyst poisoning.

The hydrogenation catalyst may be a copper chromite catalyst and preferably the catalyst further comprises zinc or iron.

According to another embodiment of the invention, the hydrogenation catalyst is a ruthenium-tin catalyst. It has been found that the hydrogenolysis of fatty esters can be carried out at lower temperatures when a ruthenium-tin catalyst is used.

The Invention will now be explained in more detail in the following non-limiting examples:

### Example 1

1-Decene, decane, 1-undecene, undecane, 1-dodecene, dodecane, 1-tridecene, tridecane, 1-tetradecene, tetradecane, 1-pentadecene, pentadecane were bought from Aldrich and used without further purification. In order to mimic a broad-cut olefin/paraffin feed stream (C₁₀-C₁₅), an olefin/paraffin mixture containing equal amounts of each of the above-mentioned olefins and paraffins was prepared (each 8.3% by volume). This feedstream has 100% by volume olefins in a 6C range (in this case C₁₀-C₁₅ olefins), in which more than 66.67% by volume of olefin molecules in the hydrocarbon stream have a total number of carbon atoms which is different from the total number of carbon atoms of the most abundant two carbon numbers of olefins in the hydrocarbon stream.

### Example 1a

### Preparation of a Range of C₁₁-C₁₆ Butyl Esters Using Cobalt/Pyridine Butoxy Carbonylation of C₁₀-C₁₅ Paraffins/α-Olefins Feedstock Followed by Fractional Distillation and Conversion of C₁₁-C₁₆ Butyl Esters to the Corresponding Alcohols

CO₂(CO)₈ (260 mg) was dissolved in 1-butanol (15 ml) and pyridine (4 ml) and heated under CO (˜80 bar) in a 50 ml autoclave to 160° C. The olefin/paraffin mixture C₁₀-C₁₅ (10 ml, pre-mixed as described above) was injected using CO (100 bar) and CO was fed at this pressure for 24 h. Olefin conversion >99%.

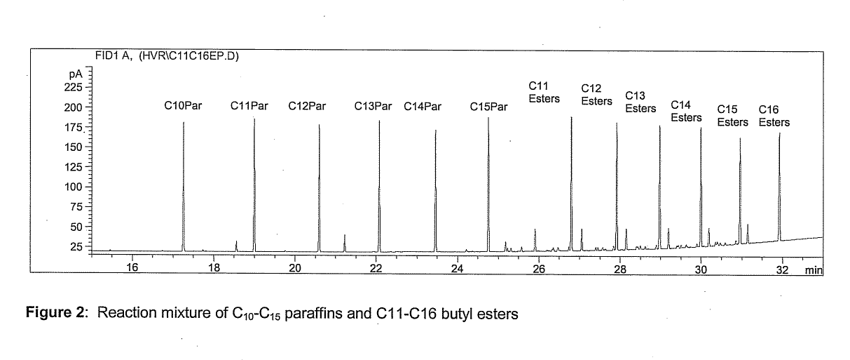

Using an Agilent Pona GC-column (50 m×0.20 mm×0.50 μsn; 40° C. for 5 minutes, 10° C./minute to 300° C., 300° C. for 20 minutes) which separates mainly on differential boiling points, the reaction mixture containing all the butyl C₁₁-C₁₆ esters and residual C₁₀-C₁₅ paraffins, was injected and the resulting GC trace provided an indication that all the C₁₀-C₁₅ paraffins could be separated via distillation from the C₁₁-C₁₆ esters, i.e. the C₁₅ paraffin has a lower boiling point than the C₁₁ esters (see FIG. 2).

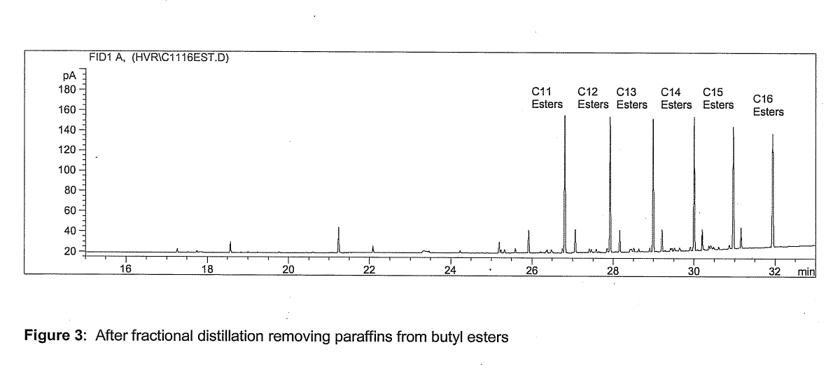

The paraffins were then removed from the reaction mixture via fractional vacuum distillation (see FIG. 3).

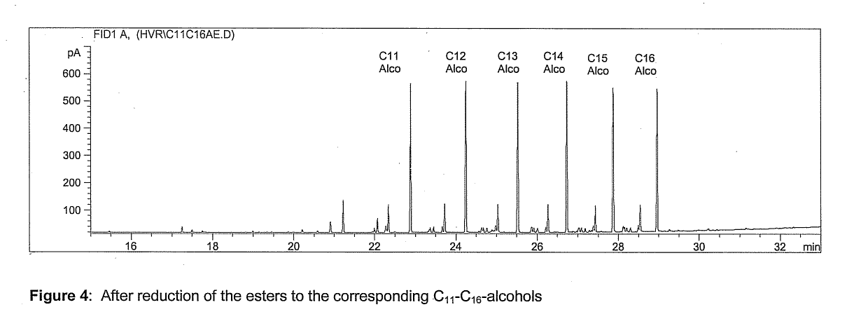

The C₁₁-C₁₆ butyl esters were then converted to the corresponding alcohols using LiAlH₄ as reducing agent (see FIG. 4). This is a non-catalytic alternative to ester H₂/catalyst hydrogenation.

### Comparative Example 1b

### Preparation of a Series of C₁₁-C₁₆ Alcohols Using Phosphine Modified Cobalt Hydroformylation of C₁₀-C₁₅ Paraffins/α-Olefins Feedstock

Co(II)-decanoate (1.2% Co in heptane) (2.5 ml, 0.36 mmol), heptane (7 ml), C₁₀-C₁₅ olefins/C₁₀-C₁₅ paraffins (20 ml, pre-mixed each 8.3% by volume as described above) and nBu₃P (1.44 mmol) were placed in a 50 ml autoclave, degassed with argon and heated to 170° C. Syngas (H₂:CO 2:1) was added to 85 bar and supplied at this pressure for 48 h.

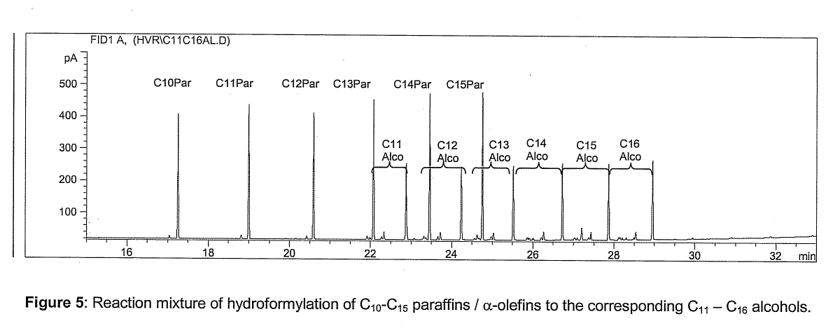

Using an Agilent Pona GC-column (50 m×0.20 mm×0.50 μsn, 40° C. for 5 minutes, 10° C./minute to 300° C., 300° C. for 20 minutes) which separates mainly on differential boiling points, the reaction mixture containing the C₁₁-C₁₆ alcohols and C₁₀-C₁₅ paraffins, was injected and the GC trace indicated that tridecane, tetradecane and pentadecane overlap with the resulting alcohols indicating that it would not be possible to separate all the paraffins via fractional distillation from the alcohol products (see FIG. 5).

### Example 2

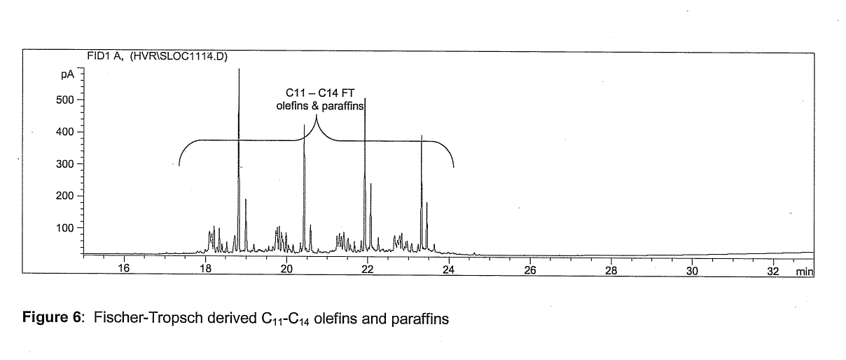

A Fischer-Tropsch derived condensate was subjected to fractional distillation to provide a C₁₁-C₁₄ feedstock with 98% by volume olefins in a 4C range (C₁₁-C₁₄), in which about 48% by volume of olefin molecules in the hydrocarbon stream have a total number of carbon atoms which is different from the total number of carbon atoms of the most abundant two (C₁₁ and C₁₂) carbon numbers of olefins in the hydrocarbon stream. The C₁₁-C₁₄ Fischer-Tropsch feedstock consists of a complex mixture of mainly olefins (C₁₀˜0.1%, C₁₁˜28%, C₁₂˜24%, C₁₃˜24%, C₁₄˜22%, C₁₅˜1.9% by volume expressed as a percentage of each carbon number class of olefins, relative to total olefin concentration) and paraffins (FIG. 6).

### Example 2a

### Preparation of a Range of C₁₂-C₁₅ Butyl Esters Using Cobalt/Pyridine 1-Butanol Hydroesterification of the Fischer-Tropsch Derived C₁₁-C₁₄ Feedstock Followed by Fractional Distillation and Conversion of C₁₂-C₁₅ Butyl Esters to the Corresponding Alcohols

CO₂(CO)₈ (260 mg) was dissolved in 1-butanol (12 ml) and pyridine (4 ml) and heated under CO (˜80 bar) in a 50 ml autoclave to 160° C. The C₁₁-C₁₄ Fischer-Tropsch derived olefin/paraffin feedstock (15 ml) was injected using CO (100 bar) and CO was fed at this pressure for 36 h.

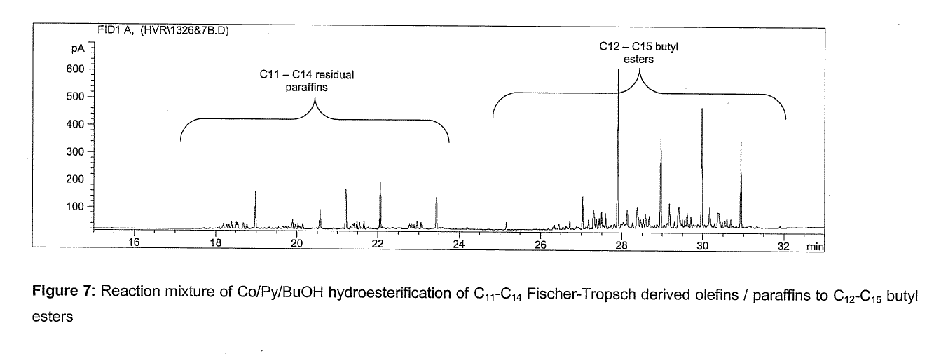

Using an Agilent Pona GC-column (50 m×0.20 mm×0.50 μsn, 40° C. for 5 minutes, 10° C./minute to 300° C., 300° C. for 20 minutes) which separates mainly on differential boiling points, the reaction mixture was injected and the resulting GC trace provided an indication that all the C₁₁-C₁₄ paraffins could be separated via distillation from the C₁₂-C₁₅ butyl esters (see FIG. 7).

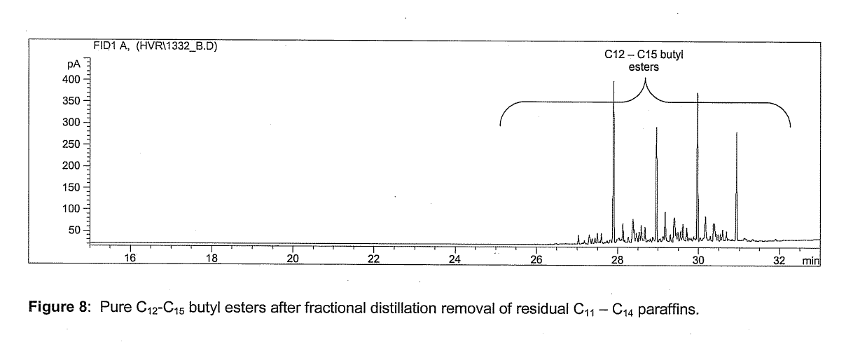

The paraffins were then removed from the reaction mixture via fractional vacuum distillation (see FIG. 8).

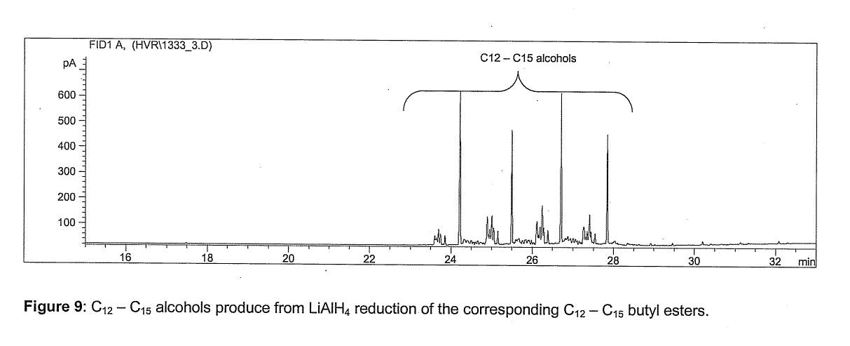

The C₁₂-C₁₅ butyl esters were then converted to the corresponding alcohols using LiAlH₄ as reducing agent affording the pure C₁₂-C₁₅ alcohols (see FIG. 9).

### Comparative Example 2b

### Preparation of a Range of C₁₂-C₁₅ Alcohols Using Phosphine Modified Cobalt Hydroformylation of the Fischer-Tropsch Derived C₁₁-C₁₄ Feedstock

Co(II)-decanoate (1.2% Co in heptane, 2.5 ml, 0.36 mmol), heptane (10 ml), Fischer-Tropsch derived C₁₁-C₁₄ condensate (18 ml) and nBu₃P (1.44 mmol) were placed in a 50 ml autoclave, degassed with argon and heated to 170° C. Syngas (H₂:CO 2:1) was added to 85 bar and supplied at this pressure for 40 h.

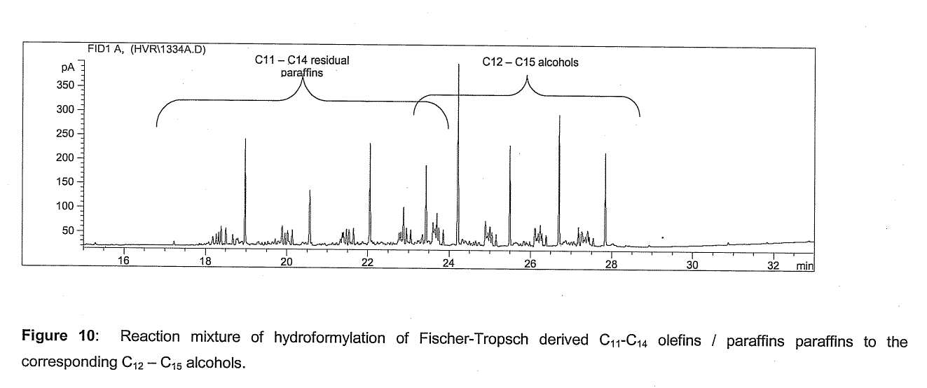

Using an Agilent Pona GC-column (50 m×0.20 mm×0.50 μsn, 40° C. for 5 minutes, 10° C./minute to 300° C., 300° C. for 20 minutes), which separates mainly on differential boiling points, the reaction mixture containing the C₁₂-C₁₅ alcohols and residual C₁₁-C₁₄ paraffins, was injected. The GC trace indicated that it would not be possible to separate all the paraffins from the required alcohols products (see FIG. 10).

1 bar=1×10⁵ Pa

We claim:

1. A process for the production of a mixture of alcohols, the process including the steps of:
1) providing a hydrocarbon stream containing olefins and paraffins in which more than 5% by volume of olefin molecules in the hydrocarbon stream have a total number of carbon atoms which is different from the total number of carbon atoms of the most abundant two carbon numbers of olefins in the hydrocarbon stream;
2) reacting the hydrocarbon stream with CO and an alcohol in the presence of a catalyst in a hydroesterification reaction to form a hydrocarbon stream containing esters and paraffins;
3) separating esters from the hydrocarbon stream containing esters and paraffins; and
4) subjecting the esters to a hydrogenation reaction to provide an alcohol product.

2. The process as claimed in claim 1, wherein the alcohols are detergent range alcohols having an average number of carbon atoms per molecule of between 8 and 20.

3. The process as claimed in claim 1, wherein the hydrocarbon stream in step 1) contains olefins in which more than 10%, by volume, of olefin molecules in the feed have a total number of carbon atoms which is different from the total number of carbon atoms of the most abundant two olefins (by carbon number) in the hydrocarbon stream.

4. The process as claimed in claim 3, wherein the hydrocarbon stream in step 1) contains olefins in which more than 20%, by volume, of olefin molecules in the feed have a total number of carbon atoms which is different from the total number of carbon atoms of the most abundant two olefins (by carbon number) in the hydrocarbon stream.

5. The process as claimed in claim 4, wherein the hydrocarbon stream in step 1) contains olefins in which more than 40%, by volume, of olefin molecules in the feed have a total number of carbon atoms which is different from the total number of carbon atoms of the most abundant two olefins (by carbon number) in the hydrocarbon stream.

6. The process as claimed in claim 1, wherein the hydrocarbon stream in step 1) contains olefins in which more than 5% by volume of olefin molecules in the feed have a total number of carbon atoms which is different from the total number of carbon atoms of the most abundant three olefins (by carbon number) in the hydrocarbon stream.

7. The process as claimed in claim 1, wherein the hydrocarbon stream in step 1) contains olefins in which more than 5% by volume of olefin molecules in the feed have a total number of carbon atoms which is different from the total number of carbon atoms of the most abundant four (by carbon number) in the hydrocarbon stream.

8. The process as claimed in claim 1, wherein the hydrocarbon stream in step 1) contains olefins in which more than 5% by volume of olefin molecules in the feed have a total number of carbon atoms which is different from the total number of carbon atoms of the most abundant five olefins (by carbon number) in the hydrocarbon stream.

9. The process as claimed in claim 1, wherein the hydrocarbon feed stream in step 1) has an average number of carbon atoms per molecule of from 10 to 18.

10. The process as claimed in claim 1, wherein the hydrocarbon feed stream in step 1) contains C₁₁-C₁₄ olefins.

11. The process as claimed in claim 1, wherein the hydrocarbon feed stream in step 1) contains C₁₀-C₁₅ olefins.

12. The process as claimed in claim 1, wherein the hydrocarbon feed stream in step 1) is derived from a Fischer-Tropsch condensate product.

13. The process as claimed in claim 1, wherein oxygenates are removed from the hydrocarbon feed stream, prior to subjecting the stream to the hydroesterification reaction of step 2).

14. The process as claimed in claim 1, wherein the esters are separated from the hydrocarbon stream containing esters, olefins and paraffins in step 3) by distillation.

15. The process as claimed in claim 1, wherein the hydroesterification reaction of step 2) is carried out in the presence of a catalyst comprising cobalt and a nitrogen-containing additive.

16. The process as claimed in claim 15, wherein the nitrogen-containing additive is pyridine.

17. The process as claimed in claim 1, wherein the hydroesterification reaction of step 2) is performed at temperatures between 120-170° C. and CO pressures between 80-150 bar (8-15×10⁶ Pa).
